(19) United States
(12) Patent Application Publication
(10) Pub. No.: US 2024/0325140 A1
(43) Pub. Date: Oct. 3, 2024

(54) INTRAOCULAR LENSES WITH ANTI-ANTERIOR CAPSULAR CONTRACTION FEATURE AND METHOD OF IMPLANTATION THEREOF

(71) Applicant: Alcon Inc., Fribourg (CH)

(72) Inventors: Terah Whiting SMILEY, Davis, CA (US); Andrew R. WALZ, Longmont, CO (US); Gregory Vinton MATTHEWS, San Francisco, CA (US); Stephen COLLINS, San Francisco, CA (US); Hanyang HUANG, Santa Clara, CA (US); William DOLLA, Plano, TX (US); Raza Ulabdin SHAH, San Francisco, CA (US); Paul MISSEL, Arlington, TX (US)

(73) Assignee: Alcon Inc., Fribourg (CH)

(21) Appl. No.: 18/616,060

(22) Filed: Mar. 25, 2024

Related U.S. Application Data
(60) Provisional application No. 63/492,435, filed on Mar. 27, 2023.
(60) Provisional application No. 63/492,430, filed on Mar. 27, 2023.

(51) Int. Cl. A61F 2/16

(52) U.S. Cl. CPC A61F 2/1635; A61F 2/1601; A61F 2002/1682; A61F 2002/169

(57) ABSTRACT

Disclosed herein are intraocular lenses with features that counteract the effects of anterior capsular contraction and methods for implanting such intraocular lenses. In some embodiments, the intraocular lenses can comprise an optic fluid chamber and a haptic having a haptic fluid lumen in fluid communication with the optic fluid chamber. The haptic fluid lumen can comprise a thickened anterior portion such that a thickness of the anterior haptic wall of the haptic is greater than the thickness of the posterior haptic wall. Moreover, the intraocular lenses can comprise one or more support columns disposed within the haptic fluid lumen. The support columns and/or the thickened anterior portion of the haptic can maintain a shape of the haptic fluid lumen in response to forces applied to the haptic as a result of anterior capsular contraction.

### CROSS-REFERENCE TO RELATED APPLICATION

This application claims priority to U.S. Patent Application No. 63/492,435 filed on Mar. 27, 2023 and U.S. Patent Application No. 63/492,430 filed on Mar. 27, 2023, the contents of which are incorporated herein by reference in their entireties.

### TECHNICAL FIELD

The present disclosure relates generally to the field of intraocular lenses, and, more specifically, to accommodating intraocular lenses.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the native crystalline lens matrix from the patient's capsular bag and replaces it with an intraocular lens (IOL). Traditional IOLs provide one or more selected focal lengths that allow the patient to have distance vision. However, after cataract surgery, patients with traditional IOLs often require glasses or other corrective eyewear for certain activities since the eye can no longer undertake accommodation (or change its optical power) to maintain a clear image of an object or focus on an object as its distance varies.

Newer IOLs such as accommodating IOLs, allow the eye to regain at least some focusing ability. Accommodating IOLs (AIOLs) use forces available in the eye to change some portion of the optical system in order to refocus the eye on distant or near targets.

AIOLs are normally implanted or introduced into a patient's capsular bag after a native lens has been removed from the capsular bag. The patient's capsular bag is connected to zonule fibers which are connected to the patient's ciliary muscles. The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flatter, which reduces the power of the lens, allowing for distance vision. When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved, which gives the lens more power, allowing the eye to focus on near objects.

An AIOL implanted within the capsular bag can also possess mechanisms that allow for the base power of the AIOL to increase when the ciliary muscles contract and decrease when the ciliary muscles relax. For example, when the ciliary muscles contract, a peripheral region of the capsular bag can apply radially inward forces on the radially outer portion of the haptic of the AIOL. The radially outer portion of the haptic can then deform and this deformation can cause the volume of a haptic fluid chamber within the haptic to decrease. When the volume of the haptic fluid chamber decreases, the fluid within the haptic fluid chamber is pushed or otherwise displaced into an optic fluid chamber within an optic portion of the AIOL. In response to this fluid entering the optic fluid chamber, the optic portion of the AIOL can change shape (e.g., increase its curvature). This shape change can allow a patient with the implanted AIOL to focus on near objects.

However, in some cases, following the implantation of an IOL, and particularly an AIOL, the patient's refractive outcome can be affected by the patient's own biological healing response. For example, fibrotic tissue can develop in areas surrounding part of the capsular bag.

While careful capsule polishing prior to IOL introduction and/or an aggressive post-operative steroid regimen can pre-empt or otherwise alleviate the build-up of fibrotic tissue, ensuring compliance with these pre-emptive measures can be challenging.

Therefore, a solution is needed which addresses the above concerns without the patient having to undergo additional surgery.

### SUMMARY

Disclosed herein are intraocular lenses with one or more features that counteract the effects of anterior capsular contraction and methods for implanting such intraocular lenses. In one embodiment, an intraocular lens is disclosed comprising an optic portion comprising an optic fluid chamber and a haptic having a proximal end coupled to the optic portion and a distal free end. The haptic comprises a haptic fluid lumen in fluid communication with the optic fluid chamber. The haptic fluid lumen extends through at least part of the haptic and is surrounded by a radially-outer haptic wall, a radially-inner haptic wall, an anterior haptic wall, and a posterior haptic wall. The thickness of the radially-inner haptic wall can be greater than the thickness of the radially-outer haptic wall in a radial direction. Moreover, the thickness of the anterior haptic wall can be greater than the thickness of the posterior haptic wall.

In some embodiments, the thickness of the anterior haptic wall can be greater than the thickness of the posterior haptic wall at a corresponding (i.e., the same) radial position relative to the optic portion.

In certain embodiments, the thickness of the anterior haptic wall can be greater than the thickness of the posterior haptic wall at a first radial position relative to the optic portion. In these embodiments, the thickness of the anterior haptic wall can be greater than the thickness of the posterior haptic wall at a second radial position relative to the optic portion. The second radial position can be radially closer to the optic portion than the first radial position.

The thickness of the anterior haptic wall can refer to an anteroposterior thickness of the anterior haptic wall as measured in an anterior-to-posterior direction. For example, the thickness of the anterior haptic wall can be measured from an anterior lumen wall surface to an anterior-most point along an exterior surface of the anterior haptic wall. Also, for example, the thickness of the posterior haptic wall can be measured from a posterior lumen wall surface to a posterior-most point along an exterior surface of the posterior haptic wall.

In other embodiments, the thickness of the anterior haptic wall can refer to an orthogonal thickness as measured in an orthogonal direction.

The thickness of the anterior haptic wall can be greater than the thickness of the radially-outer haptic wall.

In some embodiments, the anterior haptic wall, the posterior haptic wall, and the rest of the haptic can be made of the same polymeric material. In other embodiments, the anterior haptic wall can be made of a different material than the rest of the haptic including the posterior haptic wall.

The radially-inner haptic wall can taper in shape as the radially-inner haptic wall gets closer (in a radial direction) to the optic portion.

Also disclosed is an intraocular lens comprising an optic portion comprising an optic fluid chamber and a haptic having a proximal end coupled to the optic portion and a distal free end. The haptic can comprise a haptic fluid lumen extending through at least part of the haptic and in fluid communication with the optic fluid chamber. A cross-sectional profile of the haptic at a position along the haptic fluid lumen can be asymmetric such that an anterior profile of the cross-sectional profile is not the same as a posterior profile of the cross-sectional profile.

Disclosed herein is another embodiment of an intraocular lens having a haptic feature that counteracts the effects of anterior capsular contraction. The intraocular lens can comprise an optic portion comprising an optic fluid chamber and a haptic having a proximal end coupled to the optic portion and a distal free end. The haptic can comprise a haptic fluid lumen extending through at least part of the haptic and in fluid communication with the optic fluid chamber. The intraocular lens can comprise one or more support columns disposed within the haptic fluid lumen. The one or more support columns can be configured to maintain a shape of the haptic fluid lumen in response to forces applied to the haptic as a result of capsular contraction.

The one or more support columns can extend from an anterior lumen wall surface to a posterior lumen wall surface.

In some embodiments, the one or more support columns can be made of the same material as one or more walls of the haptic. In other embodiments, the one or more support columns can be made of a different material than the walls of the haptic.

The haptic fluid lumen can be surrounded by a radially-outer haptic wall, a radially-inner haptic wall, an anterior haptic wall, and a posterior haptic wall. The one or more support columns can be positioned radially closer to the radially-inner haptic wall than the radially-outer haptic wall. Moreover, the thickness of the radially-inner haptic wall can be greater than the thickness of the radially-outer haptic wall in a radial direction. In addition, the radially-inner haptic wall can taper in shape as the radially-inner haptic wall gets closer to the optic portion.

Further disclosed herein is another embodiment of an intraocular lens comprising a thickened anterior haptic portion and one or more support columns disposed within the haptic fluid lumen. The thickened anterior haptic portion and the one or more support columns can work together to maintain a shape of the haptic fluid lumen in response to forces applied to the haptic as a result of capsular contraction. The thickened anterior haptic portion can refer to a portion of an anterior haptic wall of where the thickness of the anterior haptic wall is greater than the thickness of a posterior haptic wall and a radially-outer haptic wall. The one or more support columns can be positioned radially closer to the radially-inner haptic wall than the radially-outer haptic wall.

Also disclosed is a method of implanting an intraocular lens. The method can comprise removing at least part of an anterior capsular wall of a capsular bag of a subject to form a capsulorhexis.

The method can also comprise removing cellular material from within the capsular bag using a capsular polishing tool. The capsular polishing tool can be a capsule sweep polisher. For example, the capsular polishing tool can comprise a hooked end having a flat rounded tip The step of removing the cellular material can further comprise scraping the cellular material off of an underside of a remainder of the anterior capsular wall after the capsulorhexis is formed.

The method can further comprise introducing one of the intraocular lenses disclosed herein into the capsular bag through the capsulorhexis. The intraocular lens can comprise an optic portion comprising an optic fluid chamber and a haptic having a proximal end coupled to the optic portion and a distal free end. The haptic can comprise a haptic fluid lumen extending through at least part of the haptic and in fluid communication with the optic fluid chamber. The haptic can be configured to maintain a shape of the haptic fluid lumen in response to forces applied to the haptic as a result of capsular contraction post-implantation.

### BRIEF DESCRIPTION OF THE DRAWINGS

FIG. 1A is a black-and-white image showing fibrotic anterior capsular opacification of a subject's eye caused by a build-up of fibrotic tissue.

FIG. 1B illustrates an example of fibrotic tissue applying a tensile force to an anterior portion of a capsular bag of a subject.

FIG. 1C illustrates a haptic of an IOL being deformed as a result of capsular bag contraction.

FIG. 2 illustrates a top plan view of one embodiment of an IOL having one or more anti-anterior capsular contraction (anti-ACC) features.

FIGS. 3A and 3B illustrate cross-sectional views of the IOL of FIG. 2 along cross-section A-A.

FIG. 4A illustrates a cross-sectional view of one embodiment of a haptic of an IOL having an anti-ACC feature.

FIG. 4B illustrates a cross-sectional view of another embodiment of a haptic of an IOL having an anti-ACC feature.

FIG. 4C illustrates a cross-sectional view of yet another embodiment of a haptic of an IOL having a plurality of anti-ACC features.

FIG. 5A illustrates a perspective view of one embodiment of a haptic of an IOL having the anti-ACC feature.

FIG. 5B illustrates the haptic of FIG. 5A with a distal end of the haptic removed to show a cross-section of the haptic.

FIG. 6 illustrates one embodiment of a method of implanting an IOL having one or more anti-ACC features.

FIG. 7 illustrates a capsular polishing tool extending into a capsular bag through the capsulorhexis to scrap off cellular material from an underside of the anterior capsular wall.

### DETAILED DESCRIPTION

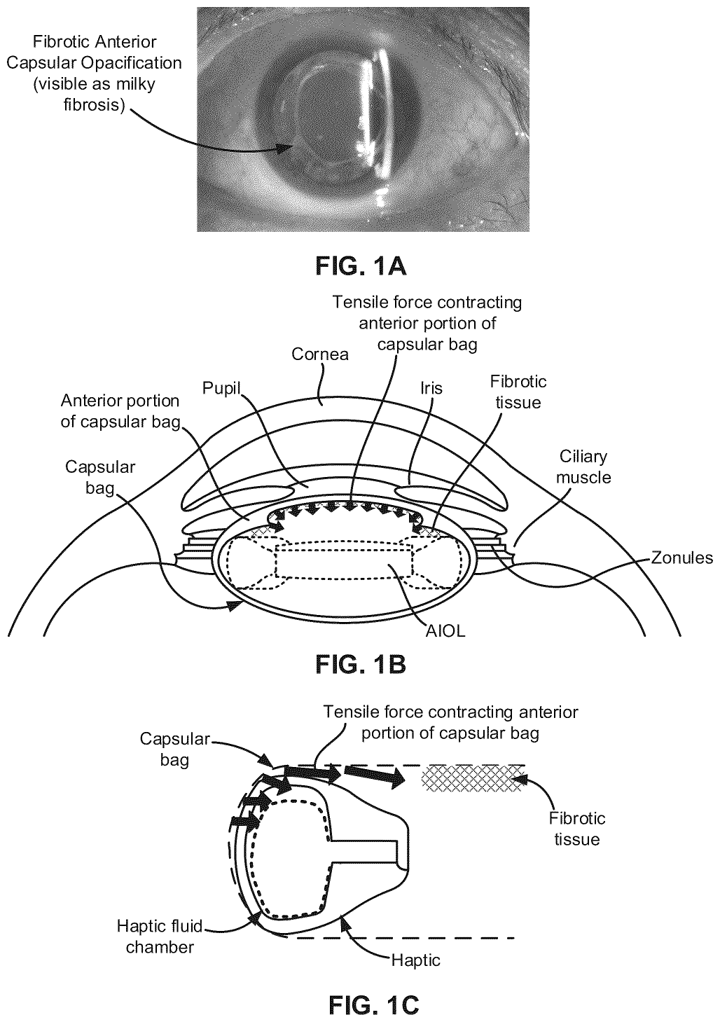

FIG. 1A is a black-and-white image showing fibrotic anterior capsular opacification (ACO) caused by the build-up of fibrotic tissue. This is also known as anterior capsular contraction (ACC), anterior capsular phimosis (AC phimosis), or capsular contraction syndrome. As shown in FIG. 1B, this fibrotic tissue can apply a tensile force to an anterior portion of the capsular bag by pulling and contracting the anterior portion of the capsular bag. FIG. 1C illustrates that this capsular bag contraction can inadvertently deform the haptic of the IOL and cause fluid within the haptic fluid chamber to be inadvertently pushed or otherwise displaced into the optic fluid chamber of the AIOL. As previously discussed, when fluid enters the optic fluid chamber, the optic portion of the IOL can change shape (e.g., increase its curvature). This inadvertent shape change can manifest itself as unwanted myopic shifts in the patient's optical power and negatively affect a patient's refractive outcome.

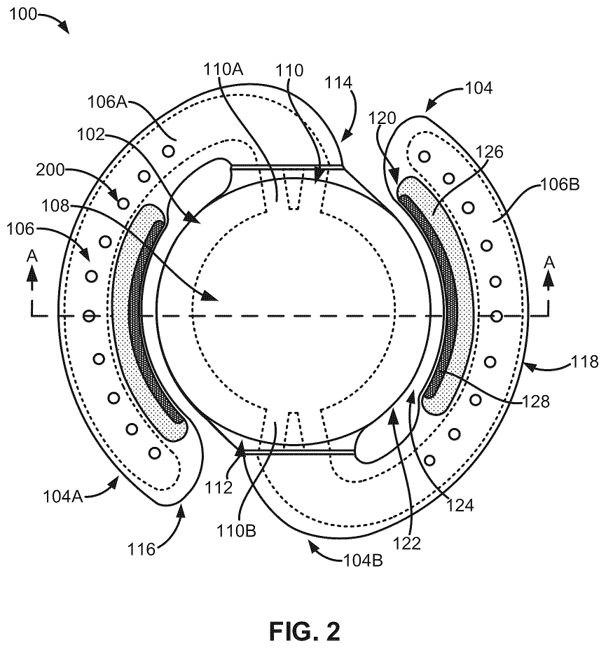

FIG. 2 illustrates a top plan view of one embodiment of an IOL 100 having one or more anti-anterior capsular contraction (anti-ACC) features. In some embodiments, the IOL 100 can be an accommodating IOL (AIOL).

Although AIOLs are depicted and described in this disclosure, any reference to an AIOL can also refer to one of the AIOLs discussed and depicted in the following U.S. publications: U.S. Pat. Pub. No. 2021/0100652; U.S. Pat. Pub. No. 2021/0100650; U.S. Pat. Pub. No. 2020/0337833; and U.S. Pat. Pub. No. 2018/0153682; and in the following issued U.S. patents: U.S. Pat. Nos. 11,426,270; 10,433,949; 10,299,913; 10,195,020; and 8,968,396, the contents of which are incorporated herein by reference in their entireties.

The IOL 100 can be implanted within a subject to correct for defocus aberration, corneal astigmatism, spherical aberration, or a combination thereof. The IOL 100 can comprise an optic portion 102 and one or more haptics 104 including a first haptic 104A and a second haptic 104B coupled to and extending peripherally from the optic portion 102. The IOL 100 can be positioned within a native capsular bag in which a native lens has been removed.

When implanted within the native capsular bag, the optic portion 102 can be adapted to refract light that enters the eye onto the retina. The one or more haptics 104 can be configured to engage the capsular bag and be adapted to deform in response to ciliary muscle movement (e.g., muscle relaxation, muscle contraction, or a combination thereof) in connection with capsular bag reshaping.

Each of the haptics 104 can comprise a haptic fluid lumen 106 extending through at least part of the haptic 104. For example, the first haptic 104A can comprise a first haptic fluid lumen 106A extending through at least part of the first haptic 104A and the second haptic 104B can comprise a second haptic fluid lumen 106B extending through at least part of the second haptic 104B. The haptic fluid lumen 106 (e.g., any of the first haptic fluid lumen 106A or the second haptic fluid lumen 106B) can be in fluid communication with or fluidly connected to an optic fluid chamber 108 within the optic portion 102.

The optic fluid chamber 108 can be in fluid communication with the one or more haptic fluid lumens 106 through one or more fluid channels 110. The fluid channels 110 can be conduits or passageways fluidly connecting the optic fluid chamber 108 to the haptic fluid lumens 106. The fluid channels 110 can be spaced apart from one another. For example, a pair of fluid channels 110 can be spaced apart between about 0.1 mm to about 1.0 mm. In some embodiments, each of the fluid channels 110 can have a diameter of between about 0.4 mm to about 0.6 mm.

The haptics 104 can be coupled to the optic portion 102 at a reinforced portion 112. The reinforced portion 112 can serve as a haptic-optic interface. The pair of fluid channels 110 can be defined or formed within part of the reinforced portion 112.

As shown in FIG. 2, the optic fluid chamber 108 can be in fluid communication with the first haptic fluid lumen 106A through a first pair of fluid channels 110A. The optic fluid chamber 108 can also be in fluid communication with the second haptic fluid lumen 106B through a second pair of fluid channels 110B.

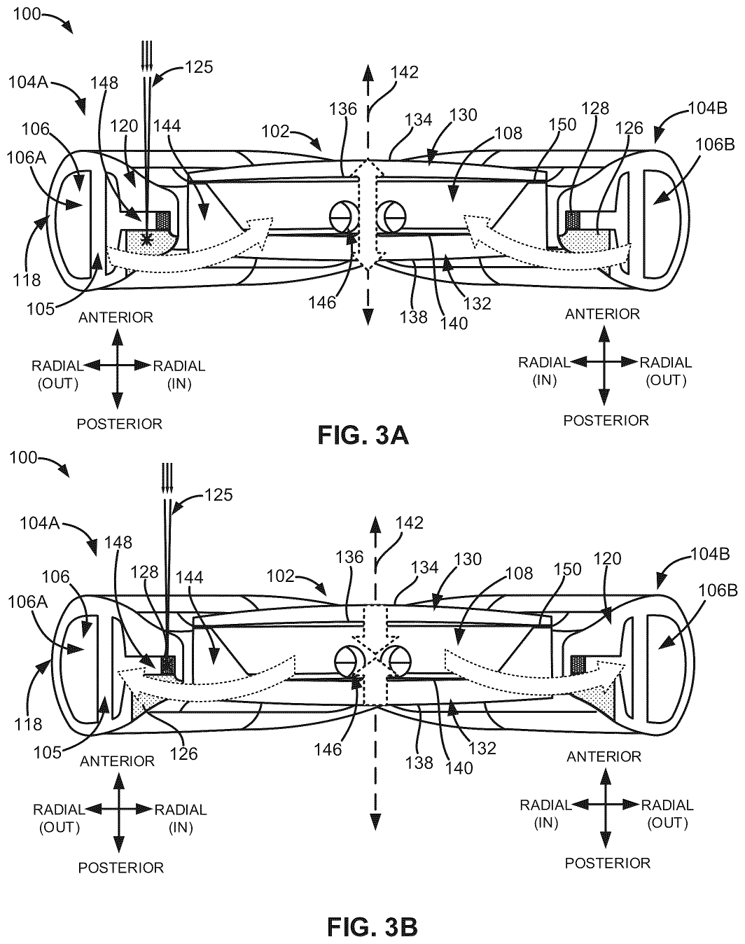

In some embodiments, the first pair of fluid channels 110A and the second pair of fluid channels 110B can be positioned substantially on opposite sides of the optic portion 102. The first pair of fluid channels 110A can be positioned substantially diametrically opposed to the second pair of fluid channels 110B. The first pair of fluid channels 110A and the second pair of fluid channels 110B can be defined or extend through part of the optic portion 102. The first pair of fluid channels 110A and the second pair of fluid channels 110B can be defined or extend through a posterior element 132 of the optic portion 102 (see, e.g., FIGS. 3A and 3B).

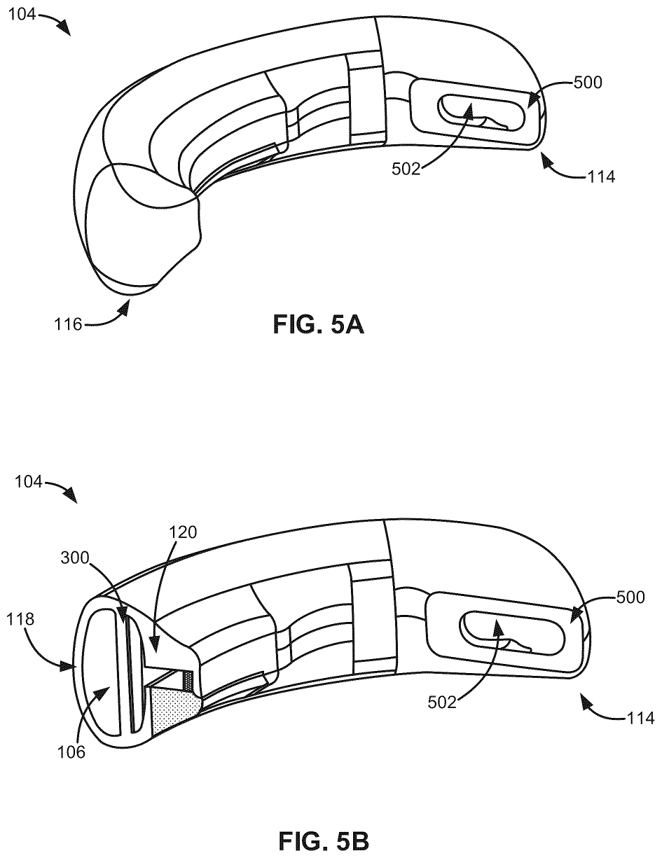

FIG. 2 also illustrates that each of the haptics 104 (e.g., any of the first haptic 104A or the second haptic 104B) can have a proximal attachment end 114 and a distal free end 116. A haptic fluid port 502 (see, e.g., FIGS. 5A and 5B) can be defined at the proximal attachment end 114 of the haptic 104. The haptic fluid port 502 can serve as an opening of the haptic fluid lumen 106. Fluid within the haptic fluid lumen 106 can flow out of the haptic fluid lumen 106 through the haptic fluid port 502 and into the optic fluid chamber 108 via the fluid channels 110 when the haptic 104 is coupled to the optic portion 102. Similarly, fluid within the optic fluid chamber 108 can flow out of the optic fluid chamber 108 through the pair of fluid channels 110 and into the haptic fluid lumen 106 through the haptic fluid port 502.

Each of the haptics 104 can comprise a radially-outer haptic wall 118 and a radially-inner haptic wall 120. The radially-outer haptic wall 118 (also referred to as a radially-outer lateral portion of the haptic 104) can be configured to face and contact an inner surface of a patient's capsular bag (see, e.g., FIG. 1B) when the IOL 100 is implanted within the capsular bag. The radially-inner haptic wall 120 (also referred to as a radially-inner lateral portion of the haptic 104) can be configured to face an outer peripheral surface 122 of the optic portion 102.

The IOL 100 can be implanted or introduced into a patient's capsular bag after a native lens has been removed from the capsular bag. The patient's capsular bag is connected to zonule fibers which are connected to the patient's ciliary muscles (see, e.g., FIG. 1B). The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flatter (in the anterior-to-posterior direction), which reduces the power of the lens, allowing for distance vision. In this configuration, the patient's native lens is said to be in a disaccommodated state or undergoing disaccommodation.

When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved (e.g., the anterior part of the lens becomes more curved), which gives the lens more power, allowing the eye to focus on near objects. In this configuration, the patient's native lens is said to be in an accommodated state or undergoing accommodation.

When the IOL 100 is implanted into a patient's capsular bag, the radially-outer haptic wall 118 of the haptics 104 can directly engage with or be in physical contact with the portion of the capsular bag that is connected to the zonules or zonule fibers. Therefore, the radially-outer haptic wall 118 can be configured to respond to capsular bag reshaping forces that are applied radially when the zonules relax and stretch as a result of ciliary muscle movements.

When the ciliary muscles contract, the peripheral region of the elastic capsular bag reshapes and applies radially inward forces on the radially-outer haptic wall 118 of each of the haptics 104. The radially-outer haptic wall 118 can then deform or otherwise changes shape and this deformation or shape-change can cause the volume of the haptic fluid lumen 106 to decrease. When the volume of the haptic fluid lumen 106 decreases, the fluid within the haptic fluid lumen 106 is moved or pushed into the optic fluid chamber 108.

The optic portion 102 can change shape in response to fluid entering the optic fluid chamber 108 from the haptic fluid lumen 106. This can increase the base power or base spherical power of the IOL 100 and allow a patient with the IOL 100 implanted within the eye of the patient to focus on near objects. In this state, the IOL 100 can be considered to have undergone accommodation.

When the ciliary muscles relax, the peripheral region of the elastic capsular bag is stretched radially outward and the capsular bag elongates and more room is created within the capsular bag. The radially-outer haptic wall 118 of the haptics 104 can be configured to respond to this capsular bag reshaping by returning to its non-deformed or non-stressed configuration. This causes the volume of the haptic fluid lumen 106 to increase or return to its non-deformed volume. This increase in the volume of the haptic fluid lumen 106 can cause the fluid within the optic fluid chamber 108 to be drawn out or otherwise flow out of the optic fluid chamber 108 and back into the haptic fluid lumen 106. As discussed previously, fluid moves out of the optic fluid chamber 108 into the haptic fluid lumen 106 through the same fluid channels 110 formed within the optic portion 102.

As previously discussed, the optic portion 102 can change shape in response to fluid exiting the optic fluid chamber 108 and into the haptic fluid lumen 106. This can decrease the base power or base spherical power of the IOL 100 and allow a patient with the IOL 100 implanted within the eye of the patient to focus on distant objects or provide for distance vision. In this state, the IOL 100 can be considered to have undergone disaccommodation.

In some embodiments, the IOL 100 can be designed such that a gap 124 or void space radially separates the radially-inner haptic wall 120 of the haptic 104 from the outer peripheral surface 122 of the optic portion 102. This can allow portions of the haptic 104 to change shape or expand in response to an external energy (e.g., laser light 125, see FIGS. 3A and 3B) directed at the haptic 104.

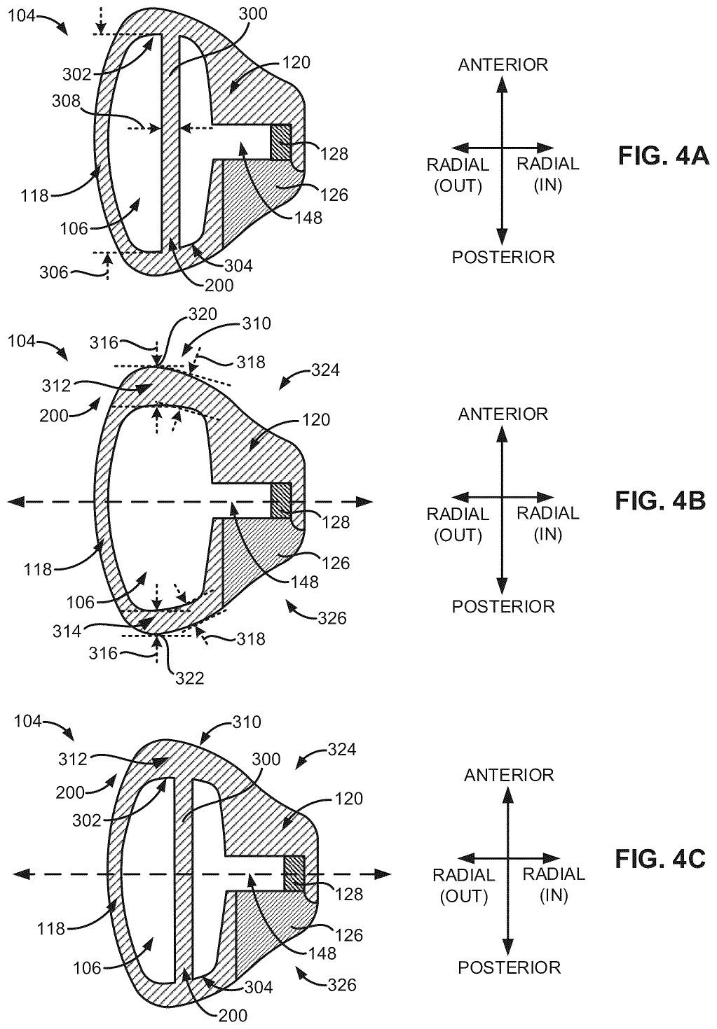

As will be discussed in more detail in the ensuing sections, each of the haptics 104 can comprise anti-ACC features 200 (e.g., support columns 300 and/or a thickened anterior portion 310, see FIGS. 4A-4C) that can counteract the effects of anterior capsular contraction caused by fibrotic tissue growth post-implantation.

FIGS. 3A and 3B illustrate cross-sectional views of the IOL 100 of FIG. 2 along cross-section A-A. The optic portion 102 can comprise an anterior element 130 and a posterior element 132. The fluid-filled optic fluid chamber 108 can be defined in between the anterior element 130 and the posterior element 132.

The anterior element 130 can comprise an anterior optical surface 134 and an anterior inner surface 136 opposite the anterior optical surface 134. The posterior element 132 can comprise a posterior optical surface 138 and a posterior inner surface 140 opposite the posterior optical surface 138. Any of the anterior optical surface 134, the posterior optical surface 138, or a combination thereof can be considered and referred to as an external optical surface. The anterior inner surface 136 and the posterior inner surface 140 can face the optic fluid chamber 108. At least part of the anterior inner surface 136 and at least part of the posterior inner surface 140 can serve as chamber walls of the optic fluid chamber 108.

As shown in FIGS. 3A and 3B, the optic portion 102 can have an optical axis 142 extending in an anterior-to-posterior direction through a center of the optic portion 102. The optical axis 142 can extend through the centers of both the anterior element 130 and the posterior element 132.

The thickness of the anterior element 130 can be greater at or near the optical axis 142 than at the periphery of the anterior element 130. In some embodiments, the thickness of the anterior element 130 can increase gradually from the periphery of the anterior element 130 toward the optical axis 142.

In certain embodiments, the thickness of the anterior element 130 at or near the optical axis 142 can be between about 0.45 mm and about 0.55 mm. In these and other embodiments, the thickness of the anterior element 130 near the periphery can be between about 0.20 mm and about 0.40 mm. Moreover, the anterior inner surface 136 of the anterior element 130 can have less curvature or be flatter than the anterior optical surface 134.

The thickness of the posterior element 132 can be greater at or near the optical axis 142 than portions of the posterior element 132 radially outward from the optical axis 142 but prior to reaching a raised periphery 144 of the posterior element 132. The thickness of the posterior element 132 can gradually decrease from the optical axis 142 to portions radially outward from the optical axis 142 (but prior to reaching the raised periphery 144). As shown in FIGS. 3A and 3B, the thickness of the posterior element 132 can increase once again from a radially inner portion of the raised periphery 144 to a radially outer portion of the raised periphery 144.

In certain embodiments, the thickness of the posterior element 132 at or near the optical axis 142 can be between about 0.45 mm and about 0.55 mm. In these and other embodiments, the thickness of the posterior element 132 radially outward from the optical axis 142 (but prior to reaching the raised periphery 144) can be between about 0.20 mm and about 0.40 mm. The thickness of the posterior element 132 near the radially outer portion of the raised periphery 144 can be between about 1.00 mm and 1.15 mm. Moreover, the posterior inner surface 140 of the posterior element 132 can have less curvature or be flatter than the posterior optical surface 138.

The optic portion 102 can have a base power or base spherical power. The base power of the optic portion 102 can be configured to change based on an internal fluid pressure within the fluid-filled optic fluid chamber 108. The base power of the optic portion 102 can be configured to increase or decrease as fluid enters or exits the fluid-filled optic fluid chamber 108.

The base power of the optic portion 102 can be configured to increase as fluid enters the fluid-filled optic fluid chamber 108 from the haptic fluid lumen(s) 106, as depicted in FIG. 3A using the curved broken-line arrows. For example, the anterior element 130 of the optic portion 102 can be configured to increase its curvature in response to the fluid entering the optic fluid chamber 108. Also, for example, the posterior element 132 of the optic portion 102 can be configured to increase its curvature in response to the fluid entering the optic fluid chamber 108. In further embodiments, both the anterior element 130 and the posterior element can be configured to increase their curvatures in response to the fluid entering the optic fluid chamber 108. These shape changes can allow a patient with the implanted IOL 100 to focus on near objects. When at least one of the anterior element 130 and the posterior element 132 has increased its curvature due to fluid entering the optic fluid chamber 108, the IOL 100 is considered to be in an accommodated state or have undergone accommodation.

The base power of the optic portion 102 can be configured to decrease as fluid exits or is drawn out of the fluid-filled optic fluid chamber 108 into the haptic fluid lumen(s) 106, as depicted in FIG. 3B using the curved broken-line arrows. For example, the anterior element 130 of the optic portion 102 can be configured to decrease its curvature (or flatten out) in response to the fluid exiting the optic fluid chamber 108. Also, for example, the posterior element 132 of the optic portion 102 can be configured to decrease its curvature (or flatten out) in response to the fluid exiting the optic fluid chamber 108. In further embodiments, both the anterior element 130 and the posterior element 132 can be configured to decrease their curvatures in response to the fluid exiting the optic fluid chamber 108. These shape changes can allow a patient with the implanted IOL 100 to focus on distant objects. When at least one of the anterior element 130 and the posterior element 132 has decreased its curvature due to fluid exiting the optic fluid chamber 108, the IOL 100 is considered to be in a disaccommodated state or has undergone disaccommodation.

It should be noted that although FIGS. 3A and 3B illustrate the fluid entering and exiting the optic fluid chamber 108 from the haptic fluid lumens 106 using the curved broken-line arrows, fluid enters and exits the optic fluid chamber 108 via the fluid channels 110 and apertures 146 defined along the posterior element 132. The apertures 146 can be holes or openings defined along the posterior element 132 that serve as terminal ends of the fluid channels 110. When the IOL 100 comprises a pair of fluid channels 110, the pair of apertures 146 serving as ends of the fluid channels 110 can be spaced apart from one another between about 0.1 mm to about 1.0 mm.

As previously discussed, the haptics 104 can be configured to deform or otherwise change shape in response to interactions or engagement with the capsular bag of the patient when the IOL 100 is implanted within an eye of the patient. More specifically, the radially-outer haptic walls 118 of the haptics 104 can be made thinner than the radially-inner haptic walls 120 to allow the haptics 104 to maintain a high degree of sensitivity to radial forces applied to an equatorial region of the haptics 104 by capsular bag reshaping as a result of ciliary muscle movements.

As shown in FIGS. 3A and 3B, the radially-inner haptic walls 120 of the haptics 104 can be designed to be thicker or bulkier than the radially-outer haptic walls 118 to provide the haptics 104 with stiffness or resiliency in the anterior-to-posterior direction. In certain embodiments, the radially-inner haptic wall 120 can taper in shape as the radially-inner haptic wall 120 gets closer to the optic portion 102. When designed in this manner, the haptics 104 can be less sensitive to capsular bag forces applied in the anterior-to-posterior direction. For example, when capsular bag forces are applied to the haptics 104 in the anterior-to-posterior direction, less fluid movement occurs between the haptic fluid lumens 106 and the optic fluid chamber 108 than when forces are applied in the radial direction. Since less fluid movement occurs, less changes in the base power of the IOL 100 occur.

In some embodiments, the fluid within the optic fluid chamber 108 and the haptic fluid lumen(s) 106 can be an oil. More specifically, in certain embodiments, the fluid within the optic fluid chamber 108 and the haptic fluid lumen(s) 106 can be a silicone oil or fluid. For example, the fluid can be a silicone oil made in part of a diphenyl siloxane. In other embodiments, the fluid can be a silicone oil made in part of a ratio of two dimethyl siloxane units to one diphenyl siloxane unit. More specifically, in some embodiments, the fluid can be a silicone oil made in part of diphenyltetramethyl cyclotrisiloxane or a copolymer of diphenyl siloxane and dimethyl siloxane. In further embodiments, the fluid can be a silicone oil comprising branched polymers.

The fluid (e.g., the silicone oil) can be index matched with a lens body material used to make the optic portion 102. When the fluid is index matched with the lens body material, the entire optic portion 102 containing the fluid can act as a single lens. For example, the fluid can be selected so that it has a refractive index of between about 1.48 and 1.53 (or between about 1.50 and 1.53). In some embodiments, the fluid (e.g., the silicone oil) can have a polydispersity index of between about 1.2 and 1.3. In other embodiments, the fluid (e.g., the silicone oil) can have a polydispersity index of between about 1.3 and 1.5. In other embodiments, the fluid (e.g., the silicone oil) can have a polydispersity index of between about 1.1 and 1.2. Other example fluids are described in U.S. Patent Publication No. 2018/0153682, which is herein incorporated by reference in its entirety.

In some embodiments, the anterior element 130 can be configured such that the anterior optical surface 134 changes shape from a spherical surface configuration to an aspherical surface configuration in response to fluid entering the optic fluid chamber 108. An aspherical surface configuration can correct for high order aberrations such as spherical aberration. The anterior optical surface 134 can be stressed into the aspherical surface configuration as a center or central portion of the anterior element 130 flexes or bulges out further than an outer periphery of the anterior element 130 which is held down by adhesives 150 or an adhesive layer.

In other embodiments, the posterior element 132 can be configured such that the posterior optical surface 138 changes shape from a spherical surface configuration to an aspherical surface configuration in response to fluid entering the optic fluid chamber 108. The posterior optical surface 138 can be stressed into the aspherical surface configuration as a center or central portion of the posterior element 132 flexes or bulges out further than an outer periphery of the anterior element 130 which is held down by adhesives 150 or the adhesive layer.

In certain embodiments, the anterior optical surface 134 can be manufactured to have an aspherical optical surface prior to the IOL 100 being implanted within the eye of the patient. In these embodiments, the anterior optical surface 134 can be aspheric regardless of any fluid pressure changes within the optic fluid chamber 108. In these embodiments, the anterior optical surface 134 can also maintain its asphericity across all base power changes.

In other embodiments, the posterior optical surface 138 can be manufactured to have an aspherical optical surface prior to the IOL 100 being implanted within the eye of the patient. In these embodiments, the posterior optical surface 138 can be aspheric regardless of any fluid pressure changes within the optic fluid chamber 108. In these embodiments, the posterior optical surface 138 can maintain its asphericity across all base power changes.

The optic portion 102 can be made in part of a deformable or flexible material. In some embodiments, the optic portion 102 can be made in part of a deformable or flexible polymeric material. For example, the anterior element 130, the posterior element 132, or a combination thereof can be made in part of a deformable or flexible polymeric material. The one or more haptics 104 (e.g., the first haptic 104A, the second haptic 104B, or a combination thereof) can be made in part of the same deformable or flexible material as the optic portion 102. In other embodiments, the one or more haptics 104 can be made in part of different materials from the optic portion 102.

In some embodiments, the optic portion 102 can comprise or be made in part of a lens body material. The lens body material can be made in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably unless otherwise specified. The cross-linked copolymer used to make the lens body material can comprise an alkyl acrylate in the amount of about 3% to 20% (wt %), a fluoro-alkyl acrylate in the amount of about 10% to 35% (wt %), and a phenyl-alkyl acrylate in the amount of about 50% to 80% (wt %). In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate. More specifically, the cross-linked copolymer used to make the lens body material can comprise n-butyl acrylate in the amount of about 3% to 20% (wt %) (e.g., between about 12% to 16%), trifluoroethyl methacrylate in the amount of about 10% to 35% (wt %) (e.g., between about 17% to 21%), and phenylethyl acrylate in the amount of about 50% to 80% (wt %) (e.g., between about 64% to 67%).

The final composition of the cross-linked copolymer used to make the lens body material can also comprise a cross-linker or cross-linking agent such as ethylene glycol dimethacrylate (EGDMA). For example, the final composition of the cross-linked copolymer used to make the lens body material can also comprise a cross-linker or cross-linking agent (e.g., EGDMA) in the amount of about 1.0%. The final composition of the cross-linked copolymer used to make the lens body material can also comprise an initiator or initiating agent (e.g., Perkadox 16) and a UV absorber.

The one or more haptics 104 can comprise or be made in part of a haptic material. The haptic material can comprise or be made in part of a cross-linked copolymer comprising a copolymer blend. The copolymer blend can comprise an alkyl acrylate, a fluoro-alkyl acrylate, and a phenyl-alkyl acrylate. For example, the cross-linked copolymer used to make the haptic material can comprise an alkyl acrylate in the amount of about 10% to 25% (wt %), a fluoro-alkyl acrylate in the amount of about 10% to 35% (wt %), and a phenyl-alkyl acrylate in the amount of about 50% to 80% (wt %). In some embodiments, the cross-linked copolymer used to make the haptic material can comprise n-butyl acrylate in the amount of about 10% to 25% (wt %) (e.g., between about 19% to about 23%), trifluoroethyl methacrylate in the amount of about 10% to 35% (wt %) (e.g., between about 14% to about 18%), and phenylethyl acrylate in the amount of about 50% to 80% (wt %) (e.g., between about 58% to about 62%). The final composition of the cross-linked copolymer used to make the haptic material can also comprise a cross-linker or cross-linking agent, such as EGDMA, in the amount of about 1.0%. The final composition of the cross-linked copolymer used to make the haptic material can also comprise a number of photoinitiators or photoinitiating agents (e.g., camphorquinone, 1-phenyl-1,2-propanedione, and 2-ethylhexyl-4-(dimenthylamino)benzoate).

In some embodiments, the refractive index of the lens body material can be between about 1.48 and about 1.53. In certain embodiments, the refractive index of the lens body material can be between about 1.50 and about 1.53 (e.g., about 1.5178).

The anterior element 130 can be attached or otherwise adhered to the posterior element 132 via adhesives 150 or an adhesive layer. The adhesive layer can be substantially annular-shaped. The adhesives 150 or adhesive layer can be positioned at a peripheral edge of the optic portion 102 in between the anterior element 130 and the posterior element 132. For example, the adhesives 150 can be positioned on top of the raised periphery 144 of the posterior element 132.

The adhesives 150 or adhesive layer can comprise or be made in part of a biocompatible adhesive. The adhesives 150 or adhesive layer can comprise or be made in part of a biocompatible polymeric adhesive.

The adhesives 150 or adhesive layer can comprise or be made in part of a cross-linkable polymer precursor formulation. The cross-linkable polymer precursor formulation can comprise or be made in part of a copolymer blend, a hydroxyl-functional acrylic monomer, and a photoinitiator.

The copolymer blend can comprise an alkyl acrylate (e.g., n-butyl acrylate in the amount of about 41% to about 45% (wt %)), a fluoro-alkyl acrylate (e.g., trifluoroethyl methacrylate in the amount of about 20% to about 24% (wt %)), and a phenyl-alkyl acrylate (phenylethyl acrylate in the amount of about 28% to about 32% (wt %)). The hydroxyl-functional acrylic monomer can be 2-hydroxyethyl acrylate (HEA).The photoinitiator can be used to facilitate curing of the adhesive. For example, the photoinitiator can be Darocur 4265 (a 50/50 blend of diphenyl(2,4,6-trimethylbenzoyl)phosphine oxide and 2-hydroxy2-methylpropiophenone).

In some embodiments, the same adhesives 150 used to bond the anterior element 130 to the posterior element 132 can also be used to bond or affix the one or more haptics 104 to the optic portion 102.

As will be discussed in more detail in the ensuing sections, each of the haptics 104 can comprise one or more anti-ACC features 200 that can counteract the effects of anterior capsular contraction caused by fibrotic tissue growth post-implantation.

FIG. 4A illustrates a cross-sectional view of one embodiment of the haptic 104 having a support column 300 disposed within the haptic fluid lumen 106. The support column 300 can be one example of an anti-ACC feature 200. The support column 300 can be configured to maintain a shape of the haptic fluid lumen 106 in response to forces applied to the haptic 104 as a result of the capsule contracting due to fibrotic tissue growth.

The support column 300 can extend from an anterior lumen wall surface 302 to a posterior lumen wall surface 304. As shown in FIG. 4A, the support column 300 can be aligned in an anterior-to-posterior direction such that the support column 300 is substantially parallel to the optical axis 142 of the optic portion 102 (see, e.g., FIGS. 3A and 3B).

In other embodiments not shown in the figures but contemplated by this disclosure, the support column 300 can be tilted or slanted. For example, the support column 300 can be positioned at an angle with respect to the optical axis 142.

As shown in FIG. 4A, the support column 300 can be positioned radially closer to the radially-inner haptic wall 120 than the radially-outer haptic wall 118 (see also, FIGS. 3A and 3B). This can allow the support column 300 to counteract the effects of fibrotic ACC without substantially affecting the sensitivity of the radially-outer haptic wall 118 to radial forces applied to the capsular bag by ciliary muscle movements.

For example, the support column 300 (or a lateral side of the support column 300 closest to the radially-inner haptic wall 120) can be separated from the radially-inner haptic wall 120 by an inner separation distance. The support column 300 (or a lateral side of the support column 300 closest to the radially-outer haptic wall 118) can be separated from the radially-outer haptic wall 118 by an outer separation distance. The outer separation distance can be greater than the inner separation distance. In some embodiments, the outer separation distance can be between 1.5 times (1.5X) to three times (3X) greater than the inner separation distance.

For example, the support column 300 can be positioned between the radially-inner haptic wall 120 and a centerline bisecting the haptic fluid lumen 106.

In some embodiments, the support column 300 can be cylindrical-shaped. In other embodiments, the support column 300 can be shaped as an elongate cuboid or square prism, a frusto-conic, a triangular prism, or an elongate ovoid (i.e., having an oval-shaped or obround cross-section).

In some embodiments, the support column 300 can be made of the same material as the rest of the haptic 104. For example, the support column 300 can be made of the same cross-linked copolymer as the walls of the haptic 104. In certain embodiments, the support column 300 can be integrated with the rest of the haptic 104. For example, the entire haptic 104, including the support column 300, can be formed by injection molding.

In other embodiments, the support column 300 can be made of a different material from the rest of the haptic 104. In these embodiments, the support column 300 can be adhered to the anterior lumen wall surface 302 and the posterior lumen wall surface 304 after the rest of the haptic 104 is formed first.

The support column 300 can have a column height 306 and a column width 308. In some embodiments, the column height 306 can be between approximately 1.5 mm to 2.0 mm. For example, the column height 306 can be between approximately 1.75 mm to 1.85 mm.

The column width 308 can be between approximately 0.20 mm to 0.60 mm. For example, the column width 308 can be between approximately 0.30 mm to 0.50 mm. In some embodiments, the column width 308 can be less than 0.20 mm or greater than 0.60 mm depending on the width of the haptic fluid lumen 106.

As shown in FIG. 4A, the column width 308 can remain constant along a length or height of the support column 300. In other embodiments, the column width 308 can be greater near the ends of the support column 300 (i.e., closer to the anterior lumen wall surface 302 and/or the posterior lumen wall surface 304) than the rest of the support column 300. For example, the support column 300 can widen at its anterior and/or posterior ends.

Although FIG. 4A illustrates only one support column 300 within the haptic fluid lumen 106, it is contemplated by this disclosure that the haptic fluid lumen 106 can comprise a plurality of support columns 300 (e.g., between three and fourteen support columns 300) positioned along a length of the haptic fluid lumen 106 (see, e.g., FIG. 2).

FIG. 4B illustrates a cross-sectional view of another embodiment of the haptic 104 having a thickened anterior portion 310. The thickened anterior portion 310 is another example of the anti-ACC feature 200. As shown in FIG. 4B, the haptic fluid lumen 106 can be surrounded by the radially-outer haptic wall 118, the radially-inner haptic wall 120, an anterior haptic wall 312, and a posterior haptic wall 314. All four walls can connect together to form a haptic fluid chamber.

In some embodiments, the thickened anterior portion 310 can refer to a part of the anterior haptic wall 312 where a thickness of the anterior haptic wall 312 is greater than a thickness of the posterior haptic wall 314. For example, the thickened anterior portion 310 can refer to a part of the anterior haptic wall 312 where a thickness of the anterior haptic wall 312 is greater than a thickness of the posterior haptic wall 314 at a corresponding radial position (i.e., at the same radial position) relative to the optic portion 102.

The thickened anterior portion 310 can also refer to a part of the anterior haptic wall 312 where a thickness of the anterior haptic wall 312 varies in a radial direction (e.g., decreases or increases in a radial direction) but the varying thicknesses are still greater than a thickness of the posterior haptic wall 314 at a corresponding radial position (i.e., at the same radial position) relative to the optic portion 102. For example, the thickness of the anterior haptic wall 312 at a first radial position relative to the optic portion 102 can be greater than the thickness of the posterior haptic wall 314 at the same first radial position. In this example, the thickness of the anterior haptic wall 312 at a second radial position closer to the optic portion than the first radial position can be less or more than the thickness of the anterior haptic wall 312 at the first radial position but can nonetheless be greater than the thickness of the posterior haptic wall 314 at the same second radial position.

In other embodiments, the thickened anterior portion 310 can be thicker than the radially-outer haptic wall 118 (i.e., thicker than a radial thickness of the radially-outer haptic wall 118).

In certain of the aforementioned embodiments, any references to the thickness of the anterior haptic wall 312 or the thickness of the posterior haptic wall 314 can refer to an anteroposterior thickness 316 of such a wall as measured in an anterior-to-posterior direction (see, FIG. 4B). Alternatively or additionally, any references to the thickness of the anterior haptic wall 312 or the thickness of the posterior haptic wall 314 can also refer to an orthogonal thickness 318 of such a wall as measured in an orthogonal direction.

For example, FIG. 4B illustrates that the anteroposterior thickness 316 of the anterior haptic wall 312 at a first radial position is greater than the anteroposterior thickness 316 of the posterior haptic wall 314 at the same first radial position. Moreover, FIG. 4B also illustrates that the anteroposterior thickness 316 of the anterior haptic wall 312 at the first radial position is greater than the orthogonal thickness 318 of the anterior haptic wall 312 at a second radial position closer to the optic portion 102 than the first radial position. Furthermore, FIG. 4B also illustrates that the orthogonal thickness 318 of the anterior haptic wall 312 at the second radial position is greater than the orthogonal thickness 318 of the posterior haptic wall 314 at the same radial position. In this example, the thickened anterior portion 310 can comprise the part of the anterior haptic wall 312 between the first radial position and the second radial position.

In some embodiments, the anteroposterior thickness 316 of the anterior haptic wall 312 can be measured from an anterior lumen wall surface 302 to an anterior-most point 320 along an exterior surface of the anterior haptic wall 312. In these and other embodiments, the anteroposterior thickness 316 of posterior haptic wall 314 can be measured from the posterior lumen wall surface 304 to a posterior-most point 322 along an exterior surface of the posterior haptic wall 314.

In certain embodiments, the anterior haptic wall 312 and the posterior haptic wall 314 can be made of the same polymeric material. For example, the anterior haptic wall 312 and the posterior haptic wall 314 can be made of the same cross-linked copolymer as the walls of the haptic 104.

In other embodiments, at least part of the anterior haptic wall 312 can be made of a different material than the posterior haptic wall 314. For example, the thickened anterior portion 310 can be made of a different material than the rest of the haptic 104 including the posterior haptic wall 314.

As a more specific example, the thickened anterior portion 310 can be made of a stiffer polymeric material than the rest of the haptic 104 including the posterior haptic wall 314 and/or the radially-outer haptic wall 118. In certain embodiments, this can be done by adding a greater amount of cross-linkers or cross-linking agents. In other embodiments, this can be done by varying an amount of the alkyl acrylate, the fluoro-alkyl acrylate, the phenyl-alkyl acrylate, or a combination thereof.

One technical problem faced by the applicants is how to counteract the effects of fibrotic ACC without interfering with the sensitivity of the fluid-filled haptic 104 to radial forces applied by the capsular bag as a result of ciliary muscle movements. The technical solution discovered and developed by the applicants are the anti-ACC features 200 disclosed herein including the thickened anterior portion 310 and/or the support columns 300 disposed within the haptic fluid lumen 106. As previously described, the support columns 300 can be positioned radially closer to the radially-inner haptic wall 120 than the radially-outer haptic wall 118 (see also, FIGS. 3A and 3B). The thickened anterior portion 310 and/or the support columns 300 can counteract the effects of fibrotic ACC without substantially affecting the sensitivity of the radially-outer haptic wall 118 to radial forces applied to the capsular bag by ciliary muscle movements.

As can be seen in FIG. 4B, the cross-sectional profile of the haptic 104 (at a position along the haptic fluid lumen 106) is asymmetric. This asymmetry can be seen by comparing an anterior profile 324 of the cross-sectional profile of the haptic 104 against a posterior profile 326 of the haptic 104. For example, the cross-sectional profile shows that an anterior profile 324 of the haptic 104 is not the same as a posterior profile 326 of the haptic 104.

FIG. 4C illustrates a cross-sectional view of yet another embodiment of the haptic 104 having both the thickened anterior portion 310 and the support column 300 disposed within the haptic fluid lumen 106. Both the thickened anterior portion 310 and the support column 300 can be configured to maintain a shape of the haptic fluid lumen 106 in response to forces applied to the haptic 104 as a result of anterior capsular contraction due to fibrotic tissue growth.

The support column 300 can extend from a posterior lumen wall surface 304 to an anterior lumen wall surface 302 serving as an underside or posterior side of the thickened anterior portion 310. The support column 300 shown in FIG. 4C can otherwise be the same as the support column 300 shown in FIG. 4A and described with respect thereto. For example, the support column 300 can be aligned in an anterior-to-posterior direction such that the support column 300 is substantially parallel to the optical axis 142 of the optic portion 102 (see, e.g., FIGS. 3A and 3B). In other embodiments not shown in the figures but contemplated by this disclosure, the support column 300 can be tilted or slanted. Also, for example, the support column 300 can be positioned radially closer to the radially-inner haptic wall 120 than the radially-outer haptic wall 118.

Moreover, the thickened anterior portion 310 shown in FIG. 4C can be the same as the thickened anterior portion 310 shown in FIG. 4B and described with respect thereto. For example, the thickened anterior portion 310 can be a part of the anterior haptic wall 312 where the thickness of the anterior haptic wall 312 is greater than a thickness of the posterior haptic wall 314 at a corresponding radial position (i.e., at the same radial position) relative to the optic portion 102. Also, for example, the thickened anterior portion 310 can be a part of the anterior haptic wall 312 where the thickness of the anterior haptic wall 312 varies in a radial direction but the thickness of the anterior haptic wall 312 is still greater than the thickness of the posterior haptic wall 314 along the same radial direction. As a more specific example, the thickness of the anterior haptic wall 312 at a first radial position (relative to the optic portion 102) can be greater than the thickness of the posterior haptic wall 314 at the same first radial position. Moreover, the thickness of the anterior haptic wall 312 at a second radial position closer to the optic portion 102 (than the first radial position) can be less or more than the thickness of the anterior haptic wall 312 at the first radial position but can nonetheless be greater than the thickness of the posterior haptic wall 314 at the same second radial position.

FIG. 4C also illustrates that the cross-sectional profile of the haptic 104 is asymmetric. This asymmetry can be seen by comparing the anterior profile 324 of the cross-sectional profile of the haptic 104 against the posterior profile 326 of the haptic 104. For example, the cross-sectional profile shows that the anterior profile 324 of the haptic 104 is not the same as the posterior profile 326 of the haptic 104.

FIGS. 4A-4C also illustrate that part of the haptic 104 can be made of a composite material. The composite material can comprise or be made in part of an energy absorbing constituent, a plurality of expandable components, and the same cross-linked copolymer used to make the rest of the haptic 104. For example, the composite material can be any of the composite materials disclosed in U.S. Patent Publication No. US2021/0100650, the content of which is incorporated herein by reference in its entirety.

The optic portion 102 (see, e.g., FIGS. 2, 3A, and 3B) can be configured to change shape in response to an external energy (e.g., light energy 125, see FIGS. 3A and 3B) directed at a composite material making up part of the haptic 104 of the IOL 100 post-implantation. As a result, the base power of the optic portion 102 can be adjusted by directing the external energy (e.g., the light energy 125, see FIGS. 3A and 3B) at the composite material making up part of the haptic 104 after the IOL 100 has been implanted within an eye of a subject.

For example, the base power of the optic portion 102 can be configured to change between about ±0.05 D to about ±0.5 D in response to the external energy (e.g., pulses of laser light 125) directed at the composite material. The base power of the optic portion 102 can be configured to change in total between about ±1.0 D and about ±2.0 D. The change in the base power can be a persistent change.

In some embodiments, the external energy can be light energy. In these embodiments, the light energy can be a laser light 125 (see, e.g., FIGS. 3A and 3B). The laser light 125 can have a wavelength of between about 488 nm to about 650 nm. For example, the laser light 125 can be a green laser light. The green laser light can have a wavelength of about 532 nm. In other embodiments, the laser light 125 can have a wavelength of between about 946 nm to about 1120 nm. For example, the laser light 125 can have a wavelength of about 1030 nm. Also, for example, the laser light 125 can have a wavelength of about 1064 nm. In some embodiments, the laser light 125 can be emitted by a neodymium-doped yttrium aluminum garnet (Nd:YAG) laser. In other embodiments, the laser light 125 can be emitted by a femtosecond laser.

In some embodiments, the energy absorbing constituent can comprise an energy absorbing pigment or dye. For example, the energy absorbing pigment can be graphitized carbon black. Also, for example, the energy absorbing pigment can be an azo dye such as Disperse Red 1 dye. In these and other embodiments, the expandable components can be expandable microspheres comprising a thermoplastic shell and a blowing agent contained within the thermoplastic shell.

As shown in FIGS. 4A-4C, the composite material can be configured as a lumen expander 128 and a lumen space-filler 126. The lumen expander 128 can be a portion of the haptic 104 made of the composite material that is designed to expand or increase a volume of the haptic fluid lumen 106 in response to the external energy directed at the lumen expander 128. The lumen space-filler 126 can be a portion of the haptic 104 made of the composite material that is designed to decrease a volume of the haptic fluid lumen 106 in response to the external energy directed at the lumen space-filler 126.

The lumen expander 128 can be positioned within a channel 148 defined within the radially-inner haptic wall 120. The channel 148 can be fluid communication with the haptic fluid lumen 106 or be part of the haptic fluid lumen 106. The channel 148 can extend partially into the radially-inner haptic wall 120.

In some embodiments, the lumen expander 128 can be positioned at a radially inner-most part of the channel 148. As shown in FIGS. 4A-4C, the radially-inner haptic wall 120 can taper in shape as the radially-inner haptic wall 120 gets closer to the optic portion 102. The lumen expander 128 can be positioned within the channel 148 near the tapered end of the radially-inner haptic wall 120.

The volume of the haptic fluid lumen 106 can increase as the lumen expander 128 expands (thereby enlarging the channel 148) in response to the external energy directed at the lumen expander 128. This can cause fluid within the optic fluid chamber 108 (see, e.g., FIGS. 2, 3A, and 3B) to be drawn out of the optic fluid chamber 108 and into the haptic fluid lumen 106. As a result, at least one of the anterior element 130 and the posterior element 132 can decrease its curvature and the base power of the optic portion 102 can decrease in response to the external energy directed at the lumen expander 128.

The lumen space-filler 126 can serve as part of the radially-inner haptic wall 120. For example, the lumen space-filler 126 can serve as part of the radially-inner haptic wall 120 posterior to the channel 148.

The volume of the haptic fluid lumen 106 can decrease as the lumen space-filler 126 expands (thereby taking up space within the haptic fluid lumen 106) in response to the external energy directed at the lumen space-filler 126. This can cause fluid within the haptic fluid lumen 106 to be pushed or otherwise displaced into the optic fluid chamber 108 (see, e.g., FIGS. 2, 3A, and 3B). As a result, at least one of the anterior element 130 and the posterior element 132 can increase its curvature and the base power of the optic portion 102 can increase in response to the external energy directed at the lumen space-filler 126.

One technical problem faced by the applicants is how to counteract the effects of fibrotic ACC without interfering with the expandable portions of the haptic 104 made of the composite material (e.g., the lumen space-filler 126 and the lumen expander 128). The technical solution discovered and developed by the applicants are the anti-ACC features 200 disclosed herein including the thickened anterior portion 310 and/or the support columns 300 disposed within the haptic fluid lumen 106. The thickened anterior portion 310 and/or the support columns 300 (which are not made of the composite material used to make the lumen space-filler 126 and/or the lumen expander 128) can counteract the effects of fibrotic ACC without substantially affecting the ability of the lumen space-filler 126 to expand and take up space within the haptic fluid lumen 106 and the ability of the lumen expander 128 to expand and increase the volume of the haptic fluid lumen 106.

In fact, one added advantage of an IOL 100 comprising the anti-ACC features 200 and the composite material is the ability to adjust a base power of the IOL 100 post-implantation by directing an external energy (e.g., laser light 125) at the composite material (for example, by a clinician such as an ophthalmic surgeon). For example, this can be done specifically to counteract the effects of anterior capsular contraction if the anti-ACC features 200 (e.g., the thickened anterior portion 310 and the support columns 300) are not enough to counteract all of the unwanted myopic shifts caused by fibrotic tissue growth along an anterior portion of the capsular bag post-implantation.

FIG. 5A illustrates a perspective view of one embodiment of a haptic 104 of an IOL 100 comprising an anti-ACC feature 200. As shown in FIG. 5B, the support column 300 can be an example of the anti-ACC feature 200. FIG. 5B illustrates the haptic 104 with a distal end 116 of the haptic 104 removed to show a cross-section of the haptic 104.

As shown in FIGS. 5A and 5B, the proximal attachment end 114 of the haptic 104 can terminate at a substantially flat interface surface 500. The flat interface surface 500 can allow the haptic 104 to adhere or otherwise couple to a corresponding interface surface (also a flat surface) protruding from the reinforced portion 112 of the optic portion 102. In some embodiments, the haptics 104 can be coupled or adhered to the optic portion 102 via the same biocompatible adhesives 150 used to adhere the anterior element 130 to the posterior element 132.

The corresponding interface surface can extend out radially from the optic portion 102. For example, the corresponding interface surface can extend out radially beyond the outer peripheral surface 122 of the optic portion 102 (e.g., extend out radially between about 10 microns and 1.0 mm beyond the outer peripheral surface 122 of the optic portion 102).

FIGS. 5A and 5B also illustrate that the substantially flat interface surface 500 can define a haptic fluid port 502. The haptic fluid port 502 can be an opening serving as a proximal end of the haptic fluid lumen 106. When the flat interface surface 500 of the haptic 104 is coupled to the corresponding interface surface of the optic portion 102, the haptic fluid port 502 can be fluidly connected to or in fluid communication with one or more exterior-facing apertures serving as terminal ends of the fluid channels 110 (see, e.g., FIG. 2). Fluid (e.g., silicone oil) entering the optic fluid chamber 108 can exit the haptic fluid lumen 106 through the haptic fluid port 502 and into the fluid channels 110. Moreover, fluid exiting the optic fluid chamber 108 can enter the haptic fluid lumen 106 through the haptic fluid port 502.

Although FIG. 5B illustrates only one support column 300 within the haptic fluid lumen 106, it is contemplated by this disclosure that the haptic fluid lumen 106 can comprise a plurality of support columns 300 positioned along a length of the haptic fluid lumen 106 (see, e.g., FIG. 2). In some embodiments, the support columns 300 can be positioned at fixed intervals along the length of the haptic fluid lumen 106. In other embodiments, the support columns 300 can be positioned at variable distances from one another along the length of the haptic fluid lumen 106.

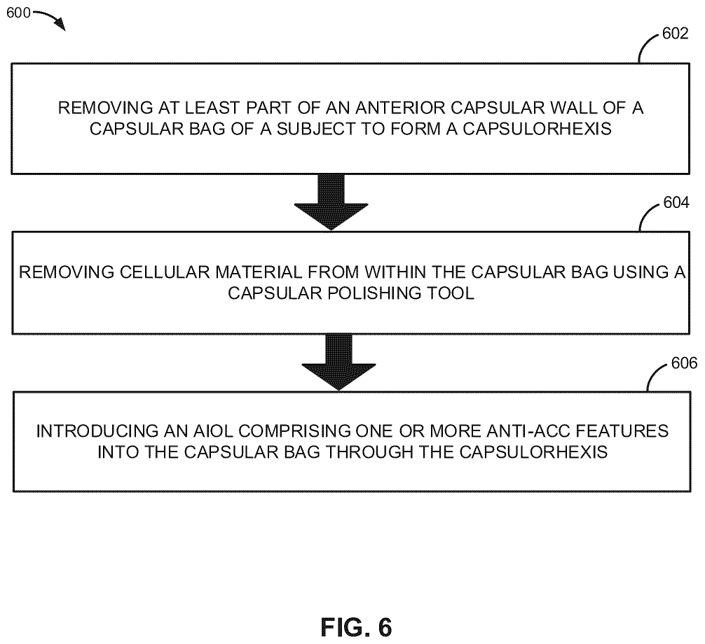

FIG. 6 illustrates one embodiment of a method 600 of implanting the IOL 100 having one or more anti-ACC features. The method 600 can comprise removing at least part of an anterior capsular wall of a capsular bag of a subject to form a capsulorhexis in step 602. The term “capsulorhexis” can refer to both the circular opening formed along the anterior capsular wall of the capsular bag or the technique of forming such an opening. In some embodiments, the capsulorhexis can be formed using a cystotome needle, capsular forceps (e.g., Utrata forceps), or a combination thereof. In other embodiments, the capsulorhexis can be formed using a femtosecond laser. The capsulorhexis can be formed as part of a phacoemulsification procedure. In certain embodiments, the phacoemulsification procedure can be any of the phacoemulsification procedures disclosed in U.S. Pat. Nos. 9,149,388; 9,119,701; and 8,784,361, the contents of which are hereby incorporated by reference in their entireties.

In other embodiments, the capsulorhexis can be formed as part of a manual small incision cataract surgery (MSICS) procedure.

The method 600 can also comprise removing cellular material from within the capsular bag using a capsular polishing tool in step 604. Step 604 can be performed after the native crystalline lens of the patient is removed. For example, step 604 can be performed after the native crystalline lens is emulsified using an ultrasonic phacoemulsification probe and aspirated from the capsular bag. The cellular material can include residual lens epithelial cells, capsular plaque, or a combination thereof leftover after the patient's native crystalline lens is removed.

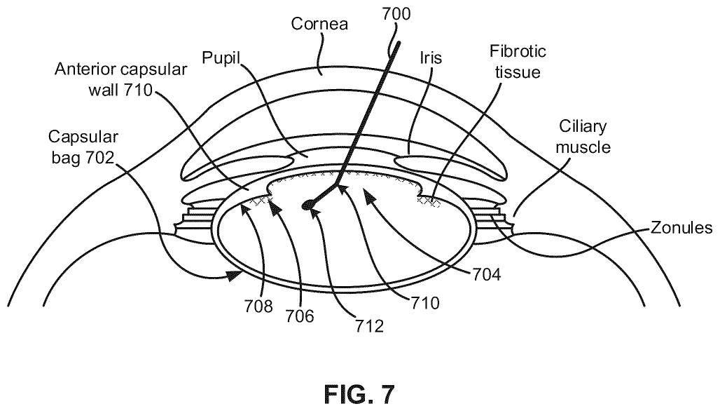

Removing the cellular material can comprise scraping the cellular material off of an underside (see, e.g., FIG. 7) of a remainder of the anterior capsular wall. For example, step 604 can comprise scraping the cellular material off of the underside of the anterior capsular wall surrounding the capsulorhexis.

In some embodiments, the capsular polishing tool can comprise a hooked end having a flat rounded tip. In certain embodiments, the flat rounded tip can be sheathed or otherwise covered by silicone. For example, the capsular polishing tool can be a capsule sweep polisher (e.g., a Singer sweep polisher).

Step 604 can further comprise irrigating the capsular bag with a viscoelastic fluid as part of the debris removal procedure and aspirating the viscoelastic fluid and any cellular material scraped off from the capsular bag using an aspirator. In certain embodiments, the capsular bag can be irrigated and aspirated using a combined irrigator-aspirator.

The method 600 can further comprise introducing the IOL 100 disclosed herein into the capsular bag through the capsulorhexis in step 606. As previously disclosed, the IOL 100 can comprise an optic portion 102 and one or more haptics 104 comprising anti-ACC features configured to maintain a shape of the haptic fluid lumen 106 in response to forces applied to the one or more haptics 104 as a result of capsular contraction post-implantation.

FIG. 7 illustrates a capsular polishing tool 700 extending into a capsular bag 702 through a capsulorhexis 704 to scrap off cellular material 706 from an underside 708 of the anterior capsular wall.

As previously discussed, the cellular material can include residual lens epithelial cells, capsular plaque, or a combination thereof leftover after the patient's native crystalline lens is removed. One technical problem faced by the applicants is that leftover cellular material (e.g., lens epithelial cells left over from the phacoemulsification procedure) can proliferate and undergo transformation into fibrotic tissue that apply tensile forces to pull and contract an anterior portion of the capsular bag. One technical solution discovered and developed by the applicants is to have a clinician (e.g., an ophthalmic surgeon) use a capsular polishing tool to scrape off the cellular material from the underside of the anterior capsular wall of the capsular bag prior to introducing an IOL comprising haptic(s) having one or more anti-ACC features into the capsular bag. Such a solution does not complicate the design of the IOL and still allows the IOL to be cost-effectively manufactured.

FIG. 7 illustrates that the capsular polishing tool 700 can comprise a hooked end 710 having a flat rounded tip 712. For example, the capsular polishing tool 700 can be a capsule sweep polisher such as a Singer sweep polisher. In certain embodiments, the flat rounded tip 712 can be sheathed or otherwise covered by silicone.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An intraocular lens, comprising:
an optic portion comprising an optic fluid chamber; and
a haptic having a proximal end coupled to the optic portion and a distal free end, the haptic comprising a haptic fluid lumen extending through at least part of the haptic and in fluid communication with the optic fluid chamber,
wherein the haptic fluid lumen is surrounded by a radially-outer haptic wall, a radially-inner haptic wall, an anterior haptic wall, and a posterior haptic wall, and
wherein a thickness of the anterior haptic wall is greater than the thickness of the posterior haptic wall.

2. The intraocular lens of claim 1, wherein the thickness of the anterior haptic wall is greater than the thickness of the posterior haptic wall at a corresponding radial position relative to the optic portion.

3. The intraocular lens of claim 1, wherein the thickness of the anterior haptic wall is greater than the thickness of the posterior haptic wall at a first radial position relative to the optic portion, wherein the thickness of the anterior haptic wall is greater than the thickness of the posterior haptic wall at a second radial position relative to the optic portion, and wherein the second radial position is radially closer to the optic portion than the first radial position.

4. The intraocular lens of claim 1, wherein the thickness refers to an anteroposterior thickness as measured in an anterior-to-posterior direction.

5. The intraocular lens of claim 4, wherein the thickness of the anterior haptic wall is measured from an anterior lumen wall surface to an anterior-most point along an exterior surface of the anterior haptic wall, and wherein the thickness of the posterior haptic wall is measured from a posterior lumen wall surface to a posterior-most point along an exterior surface of the posterior haptic wall.

6. The intraocular lens of claim 1, wherein the thickness refers to an orthogonal thickness as measured in an orthogonal direction.

7. The intraocular lens of claim 1, wherein the thickness of the anterior haptic wall is greater than the thickness of the radially-outer haptic wall.

8. The intraocular lens of claim 1, wherein the anterior haptic wall and the posterior haptic wall are made of the same polymeric material.

9. The intraocular lens of claim 1, wherein the anterior haptic wall is made of a different material than the posterior haptic wall.

10. The intraocular lens of claim 1, wherein the thickness of the radially-inner haptic wall is greater than the thickness of the radially-outer haptic wall in a radial direction.

11. The intraocular lens of claim 1, wherein the radially-inner haptic wall tapers in shape as the radially-inner haptic wall gets closer to the optic portion.

12. The intraocular lens of claim 1, further comprising one or more support columns disposed within the haptic fluid lumen, and wherein the one or more support columns are configured to maintain a shape of the haptic fluid lumen in response to forces applied to the haptic as a result of capsular contraction.

13. The intraocular lens of claim 12, wherein the one or more support columns extend from an anterior lumen wall surface to a posterior lumen wall surface.

14. The intraocular lens of claim 12, wherein the one or more support columns are made of the same material as one or more walls of the haptic.

15. The intraocular lens of claim 12, wherein the one or more support columns are positioned radially closer to the radially-inner haptic wall than the radially-outer haptic wall.

16. An intraocular lens, comprising:
an optic portion comprising an optic fluid chamber;
a haptic having a proximal end coupled to the optic portion and a distal free end, the haptic comprising a haptic fluid lumen extending through at least part of the haptic and in fluid communication with the optic fluid chamber; and
one or more support columns disposed within the haptic fluid lumen, and wherein the one or more support columns are configured to maintain a shape of the haptic fluid lumen in response to forces applied to the haptic as a result of capsular contraction.

17. The intraocular lens of claim 16, wherein the one or more support columns extend from an anterior lumen wall surface to a posterior lumen wall surface.

18. The intraocular lens of claim 16, wherein the one or more support columns are made of the same material as one or more walls of the haptic.

19. (canceled)

20. (canceled)

21. (canceled)

22. (canceled)

23. A method of implanting an intraocular lens, comprising:
removing at least part of an anterior capsular wall of a capsular bag of a subject to form a capsulorhexis;
removing cellular material from within the capsular bag using a capsular polishing tool; and
introducing the intraocular lens into the capsular bag through the capsulorhexis, wherein the intraocular lens comprises:
an optic portion comprising an optic fluid chamber; and
a haptic having a proximal end coupled to the optic portion and a distal free end, the haptic comprising a haptic fluid lumen extending through at least part of the haptic and in fluid communication with the optic fluid chamber, wherein the haptic is configured to maintain a shape of the haptic fluid lumen in response to forces applied to the haptic as a result of capsular contraction post-implantation.

24. (canceled)

25. (canceled)

26. (canceled)

27. (canceled)

28. (canceled)

29. (canceled)

30. An intraocular lens, comprising:
an optic portion comprising an optic fluid chamber, and
a haptic having a proximal end coupled to the optic portion and a distal free end, the haptic comprising a haptic fluid lumen extending through at least part of the haptic and in fluid communication with the optic fluid chamber,
wherein a cross-sectional profile of the haptic at a position along the haptic fluid lumen is asymmetric such that an anterior profile of the cross-sectional profile is not the same as a posterior profile of the cross-sectional profile.
